(19) United States
(12) Patent Application Publication
(10) Pub. No.: US 2019/0357995 A1
(43) Pub. Date: Nov. 28, 2019

(54) MEDICAL DEVICE CLEANING SYSTEM

(71) Applicant: OsteoMed LLC, Addison, TX (US)

(72) Inventors: Glenn Tage Carlson, Keller, TX (US); Benjamin Carl Casey, Dallas, TX (US)

(21) Appl. No.: 16/421,414

(22) Filed: May 23, 2019

Related U.S. Application Data
(60) Provisional application No. 62/676,826, filed on May 25, 2018.

(51) Int. Cl. A61B 90/70; B08B 3/04

(52) U.S. Cl. CPC A61B 90/70; A61B 17/142; B08B 2203/0223; B08B 3/04

(57) ABSTRACT

Systems and methods for providing a medical tool cleaning and/or lubrication system are provided. A cleaning system may include an attachment interface configured to receive one or more medical device tool modules. The attachment interface may include one or more input or output couplings which are configured to connect the attachment interface to a reservoir which provides one or more fluids in an input/output pathway for the circulation of the fluids into the one or more medical device tool modules. The cleaning system may also include one or more fluid reservoirs which contain fluid to be circulated through the one or more medical device tool modules. The cleaning system may further comprise a driver system configured to at least partially actuate components of connected medical device tool modules while the modules are being cleaned.

### CROSS-REFERENCE TO RELATED APPLICATIONS

The present application claims priority to U.S. Provisional Application No. 62/676,826 filed May 25, 2018 and entitled “MEDICAL DEVICE CLEANING SYSTEM,” the disclosure of which is incorporated by reference herein in its entirety.

### TECHNICAL FIELD

The present application relates to powered surgical tools, and more specifically to systems and methods for cleaning attachments to said tools.

### BACKGROUND

Power tools and corresponding systems are commonly used in surgical settings. Referring to FIG. 1, a power tool system 100 generally includes a control device 101 that monitors and controls various aspects of the system. The system also includes a motor portion 102 that drives a module 103 (e.g., one or more detachable components). The detachable components are generally configured to fulfill different functions. For example, a motor device may be configured to attach to one or more modules including different drill modules, reciprocating saw modules, oscillating saw modules, sagittal saw modules, wire/pin driver modules, etc. These modules often have complicated internal moving parts that are configured to translate the driving power of the motor to whatever driving force is needed for the particular module (e.g. a circulating motion, reciprocating motion, etc.). The modules are also generally configured to receive an appropriate tip (not shown) such as a blade, drill bit, grinding bur, and the like.

Because these tool systems are used in a surgical setting, it is very important that they be properly cleaned and lubricated between uses. This is important for multiple health and safety reasons. For example, without proper cleaning, an environment for bacteria and germs may be fostered in various niche areas within the internal components of the tool. Additionally, proper lubrication is important so that the tool maintains optimal performance when used in delicate surgical tasks where precision is necessary.

Currently, cleaning and lubrication of the various modules of a system (such as system 100) is generally done by soaking a respective module in various enzymatic cleaner and lubricant solutions. Other cleaning systems place the tools in dishwasher-style cleaning cycles. However, each of these types of cleaning techniques fail to completely clean the internal parts of the various modules discussed above. For example, portions of moving parts may be in contact or create dead spaces where fluids do not adequately penetrate and remove contaminants. Such areas also may not be completely lubricated for the same reasons.

### BRIEF SUMMARY

The present application describes various embodiments of systems and methods for providing a medical tool cleaning and/or lubrication system. In accordance with one embodiment, a cleaning system may include an attachment interface configured to receive one or more medical device tool modules. The attachment interface may include one or more input or output couplings which are configured to connect the attachment interface to a reservoir providing one or more fluids to an input/output pathway for the circulation of the fluids into the one or more medical device tool modules. The cleaning system may also include one or more fluid reservoirs which contain fluid to be circulated through the one or more medical device tool modules. The cleaning system may further comprise a driver system configured to at least partially actuate components of connected medical device tool modules while the modules are being cleaned.

In further embodiments, the cleaning system may be configured to be utilized with a plurality of medical device tool modules simultaneously. Embodiments may also include drain systems and/or other circulation systems to dispose of, or circulate, used fluid after it has been circulated through a module. Cleaning systems may also include various control systems which govern the duration of a cleaning cycle, enabling a system to switch between different fluids (e.g. by controlling various valves), and controlling the functionality of one or more motors configured to actuate the one or more medical device tool modules being cleaned.

Embodiments may also include methods for cleaning medical device tool modules. Such a method may comprise securing one or more modules within an attachment interface, circulating one or more fluids through an attached module, and causing the attached module to be at least partially actuated during the fluid circulation process.

In a particular embodiment, a medical tool cleaning system comprises a tool module attachment interface configured to receive at least one target tool module to be cleaned. The tool module attachment interface comprises at least one fluid inlet for receiving fluid to be propagated through the target tool module. The medical tool cleaning system also comprises a motor configured to interact with at least one received target tool module in order to actuate one or more internal components of the at least one received target tool module. The medical tool cleaning system further comprises a control system configured to initiate and stop a fluid propagation cycle and to control the interaction of the motor with the at least one received target tool module.

In some of the foregoing embodiments, the medical tool cleaning system further comprises a fluid reservoir connected to the tool module attachment interface. The fluid reservoir includes at least one fluid to be propagated through the at least one received target tool module. In some such embodiments, the fluid reservoir comprises multiple fluids to be selectively utilized to propagate through the at least one received target tool module. In some such embodiments, the multiple fluids comprise fluids for cleaning and lubrication of the at least one received target tool module.

In some of the foregoing embodiments, the motor is configured to actuate the at least one received target tool module simultaneously with the fluid being pumped through the at least one fluid inlet. Alternatively, the motor is configured to actuate the at least one received target tool module between times when the fluid is pumped through the at least one fluid inlet.

In some of the foregoing embodiments, the tool module attachment interface is configured to receive a plurality of target tool modules. In some such embodiments, the motor is configured to actuate the plurality of target tool modules. Alternatively, the medical tool cleaning system further comprises a plurality of motors, and each motor of the plurality of motors is configured to actuate a respective target tool module of the plurality of target tool modules.

In a particular embodiment, a tool module attachment interface comprises a housing defining an interior cavity configured to receive a tool module. The tool module attachment interface comprises an attachment seal configured to surround a tool module and to provide at least a partial seal between the interior cavity and an exterior environment. The tool module attachment interface also comprises a one-way valve configured to seal around a body of the tool module and to provide a fluid path through the tool module. The tool module attachment interface further comprises a dynamic seal configured to prevent fluid backflow to a motor configured to actuate one or more internal components of the tool module during a cleaning process.

In some of the foregoing embodiments, the tool module attachment interface further comprises a cavity configured to receive a driveshaft coupled to the motor. The driveshaft is configured to enable actuation of the one or more internal components of the tool module. Additionally, or alternatively, the tool module comprises one of the group of a drill module, a reciprocating saw module, an oscillating saw module, a sagittal saw module, and a wire/pin driver module. Additionally, or alternatively, the tool module attachment interface further comprises a dome switch configured to display a particular color when the tool module is inserted.

In a particular implementation, a method of medical tool cleaning comprises receiving a start command. The start command indicates that a tool module has been inserted into an attachment interface of a medical tool cleaning system. The method also comprises causing a pump to provide a first fluid to propagate through the tool module during a cleaning process. The method further comprises causing a motor to actuate one or more internal components of the tool module during the cleaning process.

In some of the foregoing embodiments, the motor actuates the one or more internal components of the tool module concurrently with propagation of the first fluid through the tool module. Additionally, or alternatively, the method further comprises receiving a user input indicating one or more settings associated with the cleaning process. The first fluid is provided, the motor is controlled, or both, in accordance with the one or more settings. Additionally, or alternatively, causing the motor to actuate the one or more internal components comprises causing the motor to actuate the one or more internal components in a first direction and causing the motor to actuate the one or more internal components in a second direction that is different from the first direction.

In some of the foregoing embodiments, the method further comprises causing the pump to drain the first fluid from the tool module. In some such embodiments, the method further comprises causing the pump to provide a second fluid to propagate through the tool module during the cleaning process. In some such embodiments, the first fluid comprises a cleaning fluid, and the second fluid comprises a lubrication fluid.

In a particular embodiment, a computer-readable storage device stores instructions that, when executed by the processor, cause the processor to perform operations comprising receiving a start command. The start command indicates that a tool module has been inserted into an attachment interface of a medical tool cleaning system. The operations also comprise sending first control signals to a pump to cause the pump to provide a first fluid to propagate through the tool module during a cleaning process. The operations further comprise sending second control signals to a motor to cause the motor to actuate one or more internal components of the tool module during the cleaning process.

In a particular embodiment, an apparatus includes means for securing a tool module. The apparatus includes means for providing a first fluid to propagate through the tool module during a cleaning process. The apparatus further includes means for actuating one or more internal components of the tool module during the cleaning process.

In a particular embodiment, a kit includes a tool attachment interface configured to secure a tool module. The tool attachment interface includes one or more seals and a valve. The kit also includes a motor configured to be coupled to the tool attachment interface and to actuate one or more internal components of the tool module during a cleaning process.

The foregoing has outlined rather broadly the features and technical advantages of the present invention in order that the detailed description that follows may be better understood. Additional features and advantages will be described hereinafter which form the subject of the claims. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the scope of the disclosure as set forth in the appended claims. The novel features which are believed to be characteristic of embodiments described herein, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present embodiments.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically. Additionally, two items that are “coupled” may be unitary with each other. To illustrate, components may be coupled by virtue of physical proximity, being integral to a single structure, or being formed from the same piece of material. Coupling may also include mechanical, thermal, electrical, communicational (e.g., wired or wireless), or chemical coupling (such as a chemical bond) in some contexts.

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. As used herein, the term “approximately” may be substituted with “within 10 percent of” what is specified. Additionally, the term “substantially” may be substituted with “within [a percentage] of” what is specified, where the percentage includes 0.1, 1, or 5 percent; or may be understood to mean with a design, manufacture, or measurement tolerance. The phrase “and/or” means and or. To illustrate, A, B, and/or C includes: A alone, B alone, C alone, a combination of A and B, a combination of A and C, a combination of B and C, or a combination of A, B, and C. In other words, “and/or” operates as an inclusive or. Similarly, the phrase “A, B, C, or a combination thereof” or “A, B, C, or any combination thereof” includes: A alone, B alone, C alone, a combination of A and B, a combination of A and C, a combination of B and C, or a combination of A, B, and C.

The terms “comprise” (and any form of comprise, such as “comprises” and “comprising”), “have” (and any form of have, such as “has” and “having”), and “include” (and any form of include, such as “includes” and “including”). As a result, an apparatus that “comprises,” “has,” or “includes” one or more elements possesses those one or more elements, but is not limited to possessing only those one or more elements. Likewise, a method that “comprises,” “has,” or “includes” one or more steps possesses those one or more steps, but is not limited to possessing only those one or more steps.

Any aspect of any of the systems, methods, and article of manufacture can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, it will be understood that the term “wherein” may be used interchangeably with “where.”

Further, a device or system that is configured in a certain way is configured in at least that way, but it can also be configured in other ways than those specifically described. The feature or features of one embodiment may be applied to other embodiments, even though not described or illustrated, unless expressly prohibited by this disclosure or the nature of the embodiments.

Some details associated with the aspects of the present disclosure are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

For a more complete understanding, reference is now made to the following descriptions taken in conjunction with the accompanying drawings, in which:

FIG. 1 illustrates an exemplary prior art surgical tool system;

FIG. 2 illustrates a tool module cleaning system in accordance with an embodiment of the present application;

FIG. 3 illustrates a cut view of a tool module attachment interface in accordance with an embodiment of the present application;

FIG. 4 illustrates a cut view of a tool module attachment interface with an inserted module in accordance with an embodiment of the present application;

FIG. 5 illustrates an embodiment of a one way valve as shown in FIGS. 3-4;

FIG. 6 illustrates a multi-module attachment interface in accordance with an embodiment of the present application;

FIG. 7 illustrates an example of a method of operating a tool module cleaning system in accordance with an embodiment of the present application; and

FIG. 8 illustrates an example of a kit including a tool attachment interface.

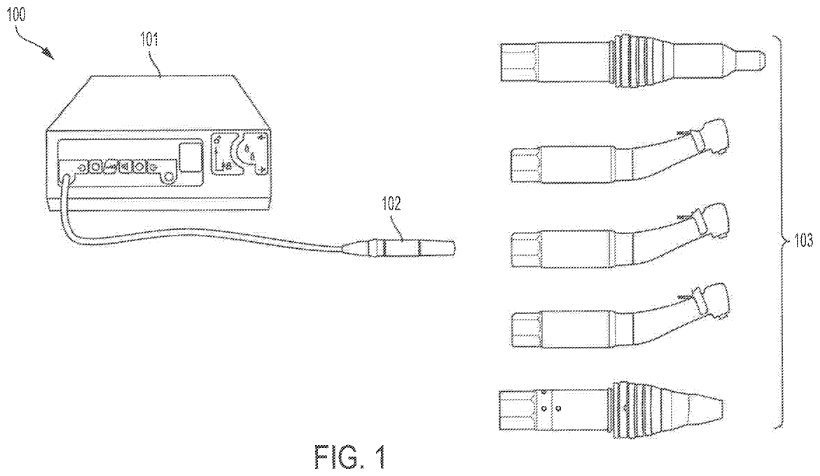

### DETAILED DESCRIPTION

The detailed description set forth below, in connection with the appended drawings, is intended as a description of various configurations and is not intended to limit the scope of the disclosure. Rather, the detailed description includes specific details for the purpose of providing a thorough understanding of the inventive subject matter. It will be apparent to those skilled in the art that these specific details are not required in every case and that, in some instances, well-known structures and components are shown in block diagram form for clarity of presentation. Likewise, the terminology used herein is for the purpose of describing particular embodiments only, and is not intended to limit the invention.

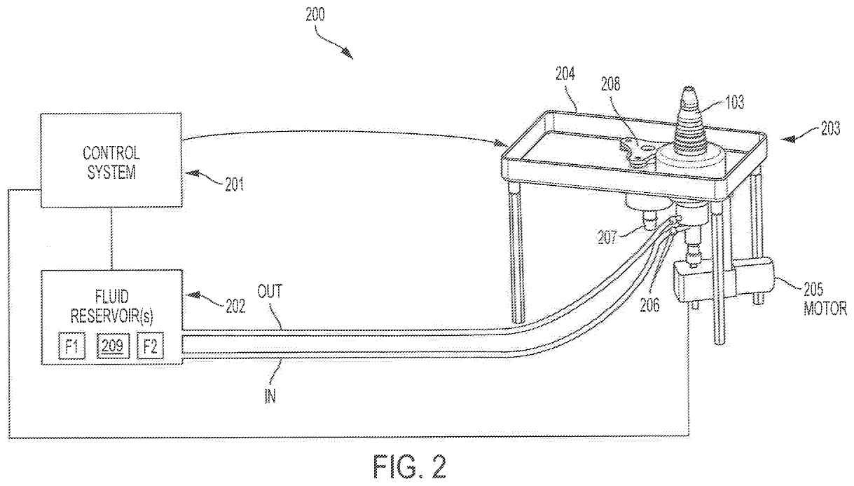

FIG. 2 illustrates a tool module cleaning system 200 in accordance with an embodiment of the present application. Cleaning system 200 includes control system 201, fluid reservoir 202, and attachment interface 203. Attachment interface 203 may include a mounting stand 204 configured to receive/mount module 103. Attachment interface 203 may also be configured to mount a motor 205. Attachment interface 203 may further include one or more fluid input/output ports 206, an output port 207 (e.g., one or more fluid return line connection points), and float valve 208. One or more fluid input/output ports 206 may be coupled to fluid reservoir 202 by one or more conduits (e.g., pipes, tubes, etc.)

Control system 201 may include various processors, memory, and input/output interfaces which enable the use and functionality of cleaning system 200. For example, in use, control system 201 may include various inputs for a user which control the type and duration of a cleaning cycle and/or a lubrication cycle to be implemented for an inserted module. When the user initiates, for example, a cleaning cycle, control system 201 sends a signal to fluid reservoir 202 which causes a pump 209 to pump fluid F1 from an outlet terminal of fluid reservoir 202 toward an input port of one or more fluid input/output ports 206 of attachment interface 203. For example, a first flow path may be established between fluid reservoir 202 and module 103 via a conduit and an input port of one or more fluid input/output ports 206. Control system 201 may also send a control signal to motor 205 which causes motor 205 to actuate internal moving components of module 103 which has been inserted for cleaning. In this manner, cleaning system 200 functions to allow fluid to be circulated through an inserted module 103 while motor 205 actuates internal components, thereby allowing for improved fluid penetration within module 103. It is appreciated that motor 205 may be utilized continuously or periodically during a fluid cycle process. Moreover, motor 205 may be controlled and configured to actuate the internal moving components of module 103 in both a forward and reverse direction. Still further, embodiments and control systems may control the functionality of motor 205 based on the type of module 103 which is inserted. For example, it is feasible that some modules may require faster spinning motions, forward and reverse spinning motions, and the like, to properly clean and lubricate the modules depending on the internal components and functionality of the respective modules. Such different cleaning processes may be preprogrammed into a memory of control system 201 or may be defined by user input.

Control system 201 may further send control signals which cause pumped fluids to either be drained and/or circulated. For example fluid reservoir 202 may include pump 209 that functions under the control of control system 201 and causes fluid to be taken in from an output port of one or more fluid input/output ports 206. For example, the first flow path established from fluid reservoir 202 to module 103 (via a conduit and an input port of one or more fluid input/output ports 206) may return to fluid reservoir 202 through module 103 and via an output port of one or more fluid input/output ports 206 and a second conduit. This fluid may be circulated back into module 103 if appropriate for the cleaning process. Alternatively, this fluid may be sent to a waste disposal drain.

It is appreciated that aspects of control system 201 may be housed within a single module or may be distributed among multiple components of cleaning system 200. For example fluid reservoir 202 may have its own pump control system which receives control signals from an external control system 201 and controls pump 209. Additionally, user inputs and outputs into control system 201 may come from communicatively coupled external computing devices such as hand-held devices or other computer terminals that are accessible to users. It is also appreciated that control system 201 may be programmed in a manner that automates some or all of a cycle, and alternatively, may allow for manual operation (e.g. by providing a start and stop switch).

It is further appreciated that control system 201 may control various types of cycles for various durations which are suitable to intended end uses. For example, in one embodiment a user may want to implement a cleaning and a lubrication cycle for an inserted module 103. In such an embodiment, control system 201 causes pump 209 to pump a first fluid F1 from fluid reservoir 202 through system 100 for a specified cleaning cycle. In this case fluid F1 may comprise enzymatic cleaner which cleans the target module. At the end of the cleaning cycle, control system 201 causes fluid F1 to be drained or otherwise evacuated from the module 103 and attachment interface 203. Thereafter, control system 201 causes second fluid F2 to be circulated through module 103 seated in attachment interface 203 for a predetermined duration of time. Second fluid F2 may comprise a lubrication fluid such as instrument milk. At the end of the lubrication cycle, control system 201 causes fluid F2 to be drained or otherwise evacuated from module 103 and attachment interface 203. Although two fluids F1 and F2 are illustrated, such illustration is not limiting, and in other implementations, fluid reservoir 202 stores one fluid or more than two fluids.

It is appreciated that various cycles, numbers and types of fluid, durations, etc., can be used within cleaning system 200. For example a cycle may simply include one cleaning fluid, or may include multiple cycles with multiple different cleaning fluids. Moreover, cycles may use different fluids, with different fluid types, and for different durations.

In another embodiment, and as shown in FIG. 2, attachment interface 203 may be configured such that mounting stand 204 is shaped to form a basin to capture fluid being cycled through module 103. Most modules, such as module 103 will have an open tip portion which is configured to receive an attachment related to a corresponding tool functionality. For example, in the event that module 103 is utilized for a drill, the tip portion of 103 will be configured to accept a drill bit. Alternatively, if module 103 is configured to implement functionality for a reciprocating saw, the tip portion will be configured to accept a saw blade. Because this tip portion is open, fluid inserted into module 103 from an input port of one or more fluid input/output ports 206 will flow out of the top portion of module 103. In such a circumstance, the basin of mounting stand 204 may function to retain circulating fluid and prevent the fluid from spilling into the environment of cleaning system 200.

Mounting stand 204 may further include a valve 208 (illustrated as a float valve). Valve 208 is configured to float and open the valve to drain the system. As described above, embodiments may include output port 207. Output port 207 may be configured to drain the basin formed in mounting stand 204. As with an output port of one or more fluid input/output ports 206, output port 207 may be connected to a waste drain or fluid exiting output port 207 may be circulated back into fluid reservoir 202 (for either recirculation or disposal).

During operation, control system 201 is configured to cause one or more cleaning cycles of module 103. To illustrate, control system 201 may receive a start command indicating that module 103 is attached to attachment interface 203. For example, a user may press a button or other control on control system 201 or communicate with control system 201 in another way, such as via a mobile device or a user input device, to indicate a start command. Control system 201 may cause pump 209 to provide a first fluid to propagate through the tool module during a cleaning process. For example, control system 201 may send first control signals to fluid reservoir 202 (or to pump 209), and pump 209 within fluid reservoir 202 may, responsive to the first control signals, cause a first fluid to be provided to an input port of one or more input/output ports 106 for propagation through module 103. Control system 201 may also cause motor 205 to actuate one or more internal components of module 103 during the cleaning process. For example, control system 201 may send second control signals to motor 205 to cause motor 205 to actuate the one or more internal components of module 103. In a particular implementation, the one or more internal components of module 103 are actuated concurrently with propagation of the first fluid through module 103.

In some implementations, the cleaning process may be performed by control system 201 in accordance with one or more settings. For example, a user may select one of one or more preprogrammed cleaning processes (e.g., cleaning processes associated with particular tools, cleaning and lubrication processes, etc.). Alternatively, control system 201 may receive a user input that indicates the one or more settings (e.g., the user may manually select the settings of the cleaning process). In some implementations, the one or more settings may include causing motor 205 to actuate the one or more internal components of module 103 in different directions (e.g., a first direction and a second direction) or at different speeds.

In a particular implementation, a medical tool cleaning system (e.g., 200) includes a tool module attachment interface (e.g., 203) configured to receive at least one target tool module (e.g., 103) to be cleaned. The tool module attachment interface includes at least one fluid inlet (e.g., 206) for receiving fluid to be propagated through the target tool module. The medical tool cleaning system includes a motor (e.g., 205) configured to interact with at least one received target tool module in order to actuate one or more internal components of the at least one received target tool module. The medical tool cleaning system also includes a control system (e.g., 201) configured to initiate and stop a fluid propagation cycle and to control the interaction of the motor with the at least one received target tool module.

Thus, FIG. 2 illustrates a cleaning system that is configured to actuate one or more internal components of module 103 during the cleaning process. Actuating the one or more internal components may cause cleaning fluid, lubrication fluid, or both to reach areas within module 103 that would not be reached if the one or more internal components were not moving. Thus, cleaning system 200 provides a more thorough cleaning and lubricating process than conventional cleaning systems or conventional lubrication systems.

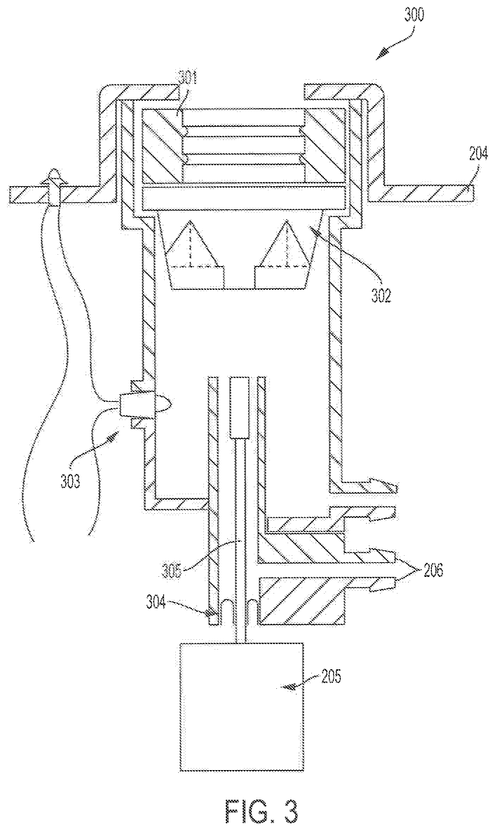

FIG. 3 illustrates a cut view of a tool module attachment interface 300 in accordance with an embodiment of the present application. Attachment interface 300 includes attachment seal 301, cross-slit, duckbill, or other one-way valve 302, dome switch 303, and dynamic seal 304. Attachment interface 300 also includes a housing that defines an internal cavity configured to receive a tool module, such as module 103. For example, attachment interface 300 includes one or more sidewalls that surround and define the interior cavity into which the tool module is to be inserted. The housing may have a first opening (e.g., a top opening, in the orientation illustrated in FIG. 3) through which an inserted tool module may extend and a second opening (e.g., a bottom opening, in the orientation illustrated in FIG. 3) through which a driveshaft 305 may extend. One or more of the seals and valves may be disposed within the internal cavity, as further described herein. Additionally, attachment interface 300 includes one or more lower sidewalls that define a driveshaft conduit configured to receive driveshaft 305 attached to motor 205. The driveshaft conduit may be located adjacent to an intended location of one or more internal components of the tool module when the tool module is inserted in attachment interface 300.

It is appreciated that various valves and seals may be utilized to ensure proper circulation of fluids through a tool module and to prevent spilling of fluids into the environment of the cleaning system. Embodiments may utilize various types of seals according to the best fit and use of the system and corresponding modules. Attachment seal 301 is configured to fit around an inserted module and provide at least a partial seal between the interior cavity of attachment interface 300 and the exterior environment. Such a seal may assist in preventing backflow of fluid and to maintain internal pressure within attachment interface 300, which allows fluids to navigate through an attached module and out of the tip of the module (or out of an output port). Attachment seal 301 may be constructed out of any suitable rubber or rubber-like material, such as silicone, and the like. Further, in some embodiments, attachment seal 301 may be implemented using a U-seal.

The cross-slit, duckbill, or other one-way valve 302 is configured to remain closed to fluid flow until a module is inserted where the valve will be held open while sealed around the body of the module The one-way valve allows for the insertion of a single module attachment in a multiple module attachment configuration without loss of cleaning fluid flow characteristics within the single module attachment.

Dome switch 303 may be configured as a depth indicating switch which shines a unique color in the upper portion visible to the user when the lower portion is tripped by the inserted module. Although illustrated in FIG. 3, dome switch 303 is optional and in some implementations is not present.

Dynamic seal 304 is disposed between motor 205 and attachment interface 300. Dynamic seal 304 functions to protect motor 205 from any fluid backflow which could leak out and damage motor 205. Additionally, dynamic seal 304 maintains fluid pressure in the internal cavity of attachment interface 300 which allows for fluid to be pumped through an attached module out through the tip of the module.

Attachment interface 300 also includes one or more fluid input/output ports 206. One or more fluid input/output ports 206 enable fluid to be provided to a tool module secured in attachment interface 300, fluid to be drained from the tool module, or both. In a particular implementation, a first fluid input/output port (e.g., the upper port in FIG. 3) may operate as an input port to provide a fluid (e.g., from fluid reservoir 202) to the interior cavity of attachment interface 300 such that the fluid propagates throughout the tool module. In a first implementation, a flow path may be established from the first fluid input/output port, through the tool module, and out a tip of the tool module (such that the fluid is received in the basin of mounting stand 204. Additionally, or alternatively, in such implementation, a second fluid input/output port (e.g., the lower port in FIG. 3) may operate as an output port to enable fluid to be drained from the tool module. Thus, a flow path may be established from the first fluid input/output port, through the tool module, and out the second fluid input/output port. In some such implementations, a portion of the fluid may flow out the tip of the tool module, and a portion of the fluid may be drained out of the second fluid input/output port.

In an alternate implementation, the second fluid input/output port may operate as an input port to provide a fluid to the interior cavity of the attachment interface 300 such that the fluid propagates through the tool module. In such implementation, a flow path may be established from the second fluid input/output port, through the tool module, and out a tip of the tool module (such that the fluid is received in the basin of mounting stand 204). Additionally, or alternatively, in such implementation, the first fluid input/output port may operate as an output port to enable fluid to be drained from the tool module. Thus, a flow path may be established from the second input/output port, through the tool module, and out the first fluid input/output port. In some such implementations, a portion of the fluid may flow out the tip of the tool module, and a portion of the fluid may be drained out of the first fluid input/output port.

In another alternate implementation, the first fluid input/output port and the second fluid input/output port may both operate as input ports to provide a fluid to the internal cavity of the attachment interface 300. In this implementation, a flow path may be established from the first fluid input/output port and the second fluid input output port, through the tool module, and out a tip of the tool module (such that the fluid is received in the basin of mounting stand 204).

In a particular implementation, a tool attachment interface includes a housing defining an interior cavity configured to receive a tool module. The tool attachment interface includes an attachment seal (e.g., 301) configured to surround the tool module and to provide at least a partial seal between the interior cavity and an exterior environment. The tool module attachment interface also includes a one-way valve (e.g., 302) configured to seal around a body of the tool module and to provide a fluid path through the tool module. The tool attachment interface further includes a dynamic seal (e.g., 304) configured to prevent backflow to a motor (e.g., 205) configured to actuate one or more internal components of the tool module during a cleaning process.

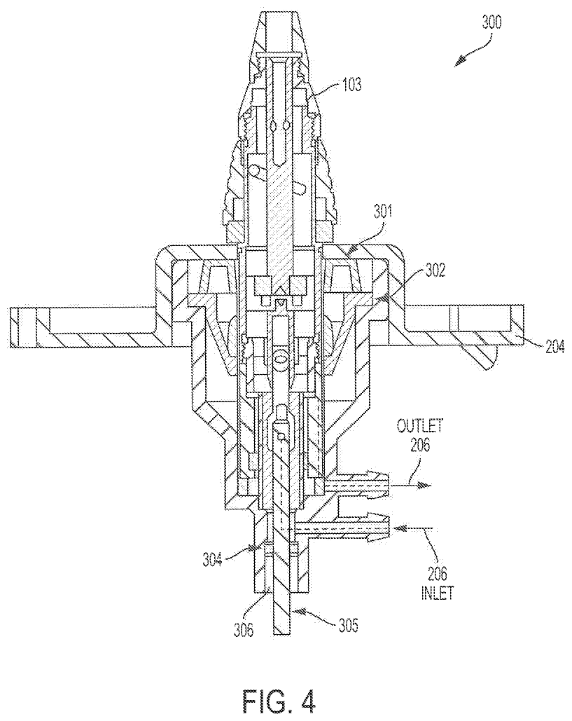

FIG. 4 illustrates cut away view of a tool module attachment interface with an inserted module in accordance with an embodiment of the present application. As illustrated, module 103 is inserted into attachment interface 300. Each of the seals and valves are engaged in order to properly seat module 103 into attachment interface 300. Additionally, driveshaft 305 which extends from motor 205 is inserted into module 103 in order to allow motor 205 to actuate the internal components of module 103. When inserted, driveshaft 305 is in contact with dynamic seal 304 and sleeve bearing 306. As can be seen, when module 103 is inserted into attachment interface 300, fluid may be introduced at an input port (e.g., an inlet port) of one or more fluid input/output ports 206 which will then propagate through the internal components of module 103 and will eventually propagate both through the tip of module 103 and through an output port (e.g., an outlet port) of one or more fluid input/output ports 206. When motor 205 is engaged and causes driveshaft 305 to rotate, fluid will propagate within various cracks and crevices and against surfaces which would not have been reached had module 103 not been in a state of actuation.

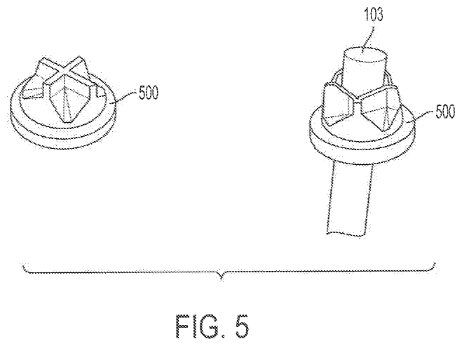

FIG. 5 illustrates an embodiment of a one way valve 500 as shown in FIGS. 3-4. As described above, the one-way valve functions to remain closed to fluid flow until a module is inserted where the valve will be held open while sealed around the body of the module.

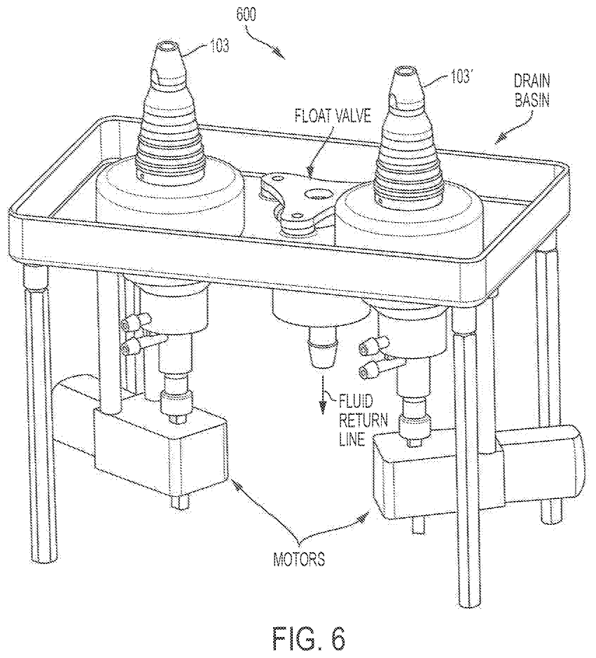

FIG. 6 illustrates a multi-module attachment interface in accordance with an embodiment of the present application. A multi-module cleaning system may function as described above with respect to a single module system. The illustrated embodiment includes two motors which correspond to a separate module. However, it is appreciated that a single motor may be utilized to provide actuation of multiple modules. In use, a cleaning cycle may be initiated which causes fluid to propagate through multiple modules 103 and 103′ and respective motors may be controlled to actuate these modules either periodically or continuously throughout the cycle. Additionally, certain control features may be altered in light of the circumstance that there are two modules present. For example, module 103 and 103′ may comprise different types of tool modules which would benefit from one or more of different duration of a cycle, amounts of fluid flow, types of motor rotation, etc. In such embodiments, control systems may be implemented in a manner where these differences are taken into account. For example, control system 201 may understand that one type of module requires forward and reverse rotation or substantially continuous rotation while another requires less. Such modifications will be understood by those skill in the art in light of the disclosure provided herein.

Embodiments of the present application may further be characterized as methods of creation and use of the cleaning and lubrication systems described above. Such methods may comprise providing and constructing the devices and systems described above. Further, a method of use may comprise inserting a tool module into an attachment mechanism, causing one or more fluids to be propagated through the attachment mechanism and corresponding module, and at least partially actuating the module during the fluid propagation. Such methods may further include initiating various additional features as describe above.

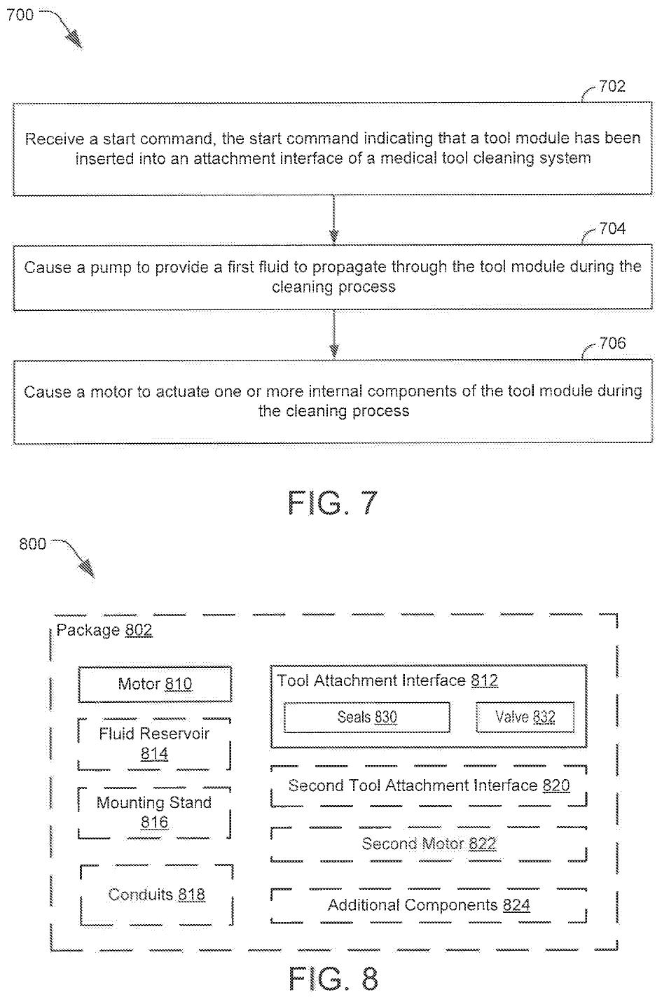

FIG. 7 illustrates a method 700 of operating a tool module cleaning system. Method 700 may be performed at or by cleaning system 200, such as by control system 201. Method 700 includes receiving a start command, at 702. The start command indicates that a tool module has been inserted into an attachment interface of a medical tool cleaning system. For example, control system 201 may receive a start command responsive to a user pressing a button on control system 201 or interfacing with control system 201 using a user input device or a mobile device.

Method 700 includes causing a pump to provide a first fluid to propagate through the tool module during a cleaning process, at 704. For example, control system 201 may send first control signals to pump 209 to provide a first fluid (e.g., F1) from fluid reservoir 202 to propagate through module 103 during the cleaning process.

Method 700 further includes causing a motor to actuate one or more internal components of the tool module during the cleaning process, at 706. For example, control system 201 may send second control signals to motor 205 to actuate one or more internal components of module 103 during the cleaning process.

In a particular implementation, the motor actuates the one or more internal components of the tool module concurrently with propagation of the first fluid through the tool module. For example, motor 205 actuates the one or more internal components of module 103 at the same time (at least partially concurrently) that the first fluid (e.g., F1) is propagated through module 103. Additionally, or alternatively, method 700 may further include receiving a user input indicating one or more settings associated with the cleaning process. The first fluid is provided, the motor is controlled, or both, in accordance with the one or more settings. For example, control system 201 may receive a user input indicating one or more settings, such as types of fluids to be used, directions or speeds of actuation, etc., and pump 209 and motor 205 may be controlled based on the one or more settings. Additionally, or alternatively, causing the motor to actuate the one or more internal components may include causing the motor to actuate the one or more internal components in a first direction and causing the motor to actuate the one or more internal components in a second direction that is different from the first direction. For example, motor 205 may be controlled to actuate the one or more internal components of module 103 in different directions, at different speeds, or both.

In a particular implementation, method 700 includes causing the pump to drain the first fluid from the tool module. For example, control system 201 may cause pump 209 to drain the first fluid (e.g., F1) from module 103. In some such implementations, method 700 further includes causing the pump to provide a second fluid to propagate through the tool module during the cleaning process. For example, control system 201 may cause pump 290 to provide a second fluid (e.g., F2) to propagate through module 103 during the cleaning process. In some such implementations, the first fluid is a cleaning fluid, and the second fluid is a lubrication fluid. For example, F1 may include enzymatic cleaner, and F2 may include instrument milk.

Thus, method 700 enables actuation of one or more internal components of a tool module while a fluid is propagated through the tool module. Propagating the fluid through the tool module while the one or more internal components are actuated enables the fluid to reach areas of the tool module that would otherwise be unreachable if the tool module were in a shut-down state. Thus, method 700 improves the effectiveness of a cleaning process, a lubrication process, or both, as compared to conventional cleaning or lubrication processes.

One or more of the methods described herein may be implemented as a computer-readable storage device storing instructions that, when executed by a processor, cause the processor to perform the operations corresponding to the method. Additionally, or alternatively, a computer-readable storage device may store instructions that, when executed by a processor, cause the processor to perform operations including receiving a start command. The start command indicates that a tool module has been inserted into an attachment interface of a medical tool cleaning system. The operations may include sending first control signals to a pump to cause the pump to provide a first fluid to propagate through the tool module during the cleaning process. The operations may further include sending second control signals to a motor to cause the motor to actuate one or more internal components of the tool module during the cleaning process.

Referring to FIG. 8, a kit 800 for medical tool cleaning systems is illustrated. Kit 800 includes motor 810, tool attachment interface 812, or both. The motor may include or correspond to the motor 205 and the tool attachment interface 812 may include or correspond to the attachment interface 203.

In some implementations, tool attachment interface 812 includes one or more seals 830 and a valve 832. One or more seals 830 may include an attachment seal configured to fit around an inserted tool module and to provide at least a partial seal between an interior cavity of tool attachment interface 812 and an outside environment, a dynamic seal configured to protect motor 810 from any fluid backflow, or both. For example, one or more seals 830 may include or correspond to attachment seal 301, dynamic seal 304, or both. Valve 832 may be configured to remain closed to fluid flow until a tool module is inserted where valve 834 will be held open while sealed around the body of the tool module. For example, valve 832 may include or correspond to cross-slit, duckbill, or other one-way valve 302 or one way valve 500. In some implementations, one or more seals 830 and valve 832 may be coupled to tool attachment interface 812. In other implementations, one or more seals 830, valve 832, or both, are separate from tool attachment interface 812 and require assembly before use.

In some implementations, kit 800 further includes a fluid reservoir 814, mounting stand 816, conduits 818, a second tool attachment interface 820, a second motor 822, and/or additional components 824. Fluid reservoir 814 is configured to store one or more fluids for providing to fluid input/output ports of tool attachment interface 812. Fluid reservoir 814 may include a pump configured to pump fluid to (or drain fluid from) tool attachment interface 812. For example, fluid reservoir 814 may include or correspond to fluid reservoir 202. Mounting stand 816 may be configured to support tool attachment interface 812 and mount tool attachment interface 812 to motor 810. In some implementations, mounting stand 816 may include a basin for receiving fluid ejected out the tip of a tool module inserted in tool attachment interface 812. Mounting stand 816 may include or correspond to mounting stand 204. Conduits 818 may include pipes, tubing, or other conduits for connecting fluid reservoir 814 to fluid input/output ports of tool attachment interface 812. Second tool attachment interface 820 may include a second tool attachment interface, similar to tool attachment interface 812. Second tool attachment interface 820 may be configured to receive a second tool module for cleaning concurrently with cleaning of a first tool module received by tool attachment interface 812. In some implementations, motor 810 may be configured to be coupled to tool attachment interface 812 and to second tool attachment interface 820. Alternatively, second tool attachment interface 820 may be connected to second motor 822. Additional components 824 include any additional components used to connect or operate one of the other components, such as conduits, adhesive, seals, clamps, or other components.

In some implementations, kit 800 may include a package 802. For example, package 802 may include a box, a bag, a container, or the like. Package 802 may include motor 810 and/or tool attachment interface 812. In some implementations, package 802 may further include fluid reservoir 814, mounting stand 816, conduits 818, second tool attachment interface 820, second motor 822, additional components 824, or any combination thereof. Additionally, or alternatively, package 802 may include a packaging medium (e.g., a packaging material), such as foam, paper, or the like. Thus, FIG. 8 describes kit 800 for a medical tool cleaning system.

In conjunction with the described aspects, an apparatus includes means for securing a tool module. The means for securing may include or correspond to attachment interface 203 of FIG. 2, attachment interface 300 of FIG. 3, tool attachment interface 812 of FIG. 8, one or more other structures configured to secure a tool module, or any combination thereof.

The apparatus also includes means for providing a first fluid to propagate through the tool module during a cleaning process. The means for providing may include or correspond to fluid reservoir 202, pump 209 of FIG. 2, one or more other structures or circuits configured to provide a first fluid to propagate through a tool module during a cleaning process, or any combination thereof.

The apparatus further includes means for actuating one or more internal components of the tool module during the cleaning process. The means for actuating may include or correspond to pump 209 of FIG. 2, driveshaft 305 of FIG. 3, one or more other structures or circuits configured to actuate one or more internal components of the tool module during the cleaning process, or a combination thereof.

It should be understood that the present system, kits, apparatuses, methods, and computer-readable storage devices are not intended to be limited to the particular forms disclosed. Rather, they are to cover all combinations, modifications, equivalents, and alternatives falling within the scope of the claims.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

The above specification and examples provide a complete description of the structure and use of illustrative examples. Although certain aspects have been described above with a certain degree of particularity, or with reference to one or more individual examples, those skilled in the art could make numerous alterations to aspects of the present disclosure without departing from the scope of the present disclosure. As such, the various illustrative examples of the methods and systems are not intended to be limited to the particular forms disclosed. Rather, they include all modifications and alternatives falling within the scope of the claims, and implementations other than the ones shown may include some or all of the features of the depicted examples. For example, elements may be omitted or combined as a unitary structure, connections may be substituted, or both. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one example or may relate to several examples. Accordingly, no single implementation described herein should be construed as limiting and implementations of the disclosure may be suitably combined without departing from the teachings of the disclosure.

In the foregoing Detailed Description, various features are grouped together in several embodiments for the purpose of streamlining the disclosure. This method of disclosure is not to be interpreted as reflecting an intention that the disclosed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

Although the embodiments of the present disclosure and their advantages have been described in detail, it should be understood that various changes, substitutions and alterations can be made herein without departing from the scope of the disclosure as defined by the appended claims. Moreover, the scope of the present disclosure is not intended to be limited to the particular embodiments of the process, machine, manufacture, composition of matter, means, methods and steps described herein. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A medical tool cleaning system comprising:
a tool module attachment interface configured to receive at least one target tool module to be cleaned, the tool module attachment interface comprising at least one fluid inlet for receiving fluid to be propagated through the target tool module;
a motor configured to interact with at least one received target tool module in order to actuate one or more internal components of the at least one received target tool module; and
a control system configured to initiate and stop a fluid propagation cycle and to control the interaction of the motor with the at least one received target tool module.

2. The medical tool cleaning system of claim 1, further comprising a fluid reservoir connected to the tool module attachment interface, the fluid reservoir including at least one fluid to be propagated through the at least one received target tool module.

3. The medical tool cleaning system of claim 2, wherein the fluid reservoir comprises multiple fluids to be selectively utilized to propagate through the at least one received target tool module.

4. The medical tool cleaning system of claim 3, wherein the multiple fluids comprise fluids for cleaning and lubrication of the at least one received target tool module.

5. The medical tool cleaning system of claim 1, wherein the motor is configured to actuate the at least one received target tool module simultaneously with the fluid being pumped through the at least one fluid inlet.

6. The medical tool cleaning system of claim 1, wherein the motor is configured to actuate the at least one received target tool module between times when the fluid is pumped through the at least one fluid inlet.

7. The medical tool cleaning system of claim 1, wherein the tool module attachment interface is configured to receive a plurality of target tool modules.

8. The medical tool cleaning system of claim 7, wherein the motor is configured to actuate the plurality of target tool modules.

9. The medical tool cleaning system of claim 7, further comprising a plurality of motors, each motor of the plurality of motors configured to actuate a respective target tool module of the plurality of target tool modules.

10. A tool module attachment interface comprising:
a housing defining an interior cavity configured to receive a tool module;
an attachment seal configured to surround the tool module and to provide at least a partial seal between the interior cavity and an exterior environment;
a one-way valve configured to seal around a body of the tool module and to provide a fluid path through the tool module; and
a dynamic seal configured to prevent fluid backflow to a motor configured to actuate one or more internal components of the tool module during a cleaning process.

11. The tool module attachment interface of claim 10, further comprising a cavity configured to receive a driveshaft coupled to the motor, the driveshaft configured to enable actuation of the one or more internal components of the tool module.

12. The tool module attachment interface of claim 10, wherein the tool module comprises one of the group of a drill module, a reciprocating saw module, an oscillating saw module, a sagittal saw module, and a wire/pin driver module.

13. The tool module attachment interface of claim 10, further comprising a dome switch configured to display a particular color when the tool module is inserted.

14. A method of medical tool cleaning, the method comprising:
receiving a start command, the start command indicating that a tool module has been inserted into an attachment interface of a medical tool cleaning system;
causing a pump to provide a first fluid to propagate through the tool module during a cleaning process; and
causing a motor to actuate one or more internal components of the tool module during the cleaning process.

15. The method of claim 14, wherein the motor actuates the one or more internal components of the tool module concurrently with propagation of the first fluid through the tool module.

16. The method of claim 14, further comprising receiving a user input indicating one or more settings associated with the cleaning process, wherein the first fluid is provided, the motor is controlled, or both, in accordance with the one or more settings.

17. The method of claim 14, wherein causing the motor to actuate the one or more internal components comprises:
causing the motor to actuate the one or more internal components in a first direction; and
causing the motor to actuate the one or more internal components in a second direction that is different from the first direction.

18. The method of claim 14, further comprising causing the pump to drain the first fluid from the tool module.

19. The method of claim 18, further comprising causing the pump to provide a second fluid to propagate through the tool module during the cleaning process.

20. The method of claim 19, wherein the first fluid comprises a cleaning fluid, and wherein the second fluid comprises a lubrication fluid.
